(19) United States
(12) Patent Application Publication
(10) Pub. No.: US 2022/0304800 A1
(43) Pub. Date: Sep. 29, 2022

(54) Valve Reshaping Device, System, and Related Methods

(71) Applicant: Kar Health, LLC, Iowa City, IA (US)

(72) Inventors: Wassef Karrowni, Cedar Rapids, IA (US); Mirna Karouni, Cedar Rapids, IA (US)

(21) Appl. No.: 17/701,931

(22) Filed: Mar. 23, 2022

Related U.S. Application Data
(60) Provisional application No. 63/164,701, filed on Mar. 23, 2021.

(51) Int. Cl. A61F 2/24

(52) U.S. Cl. CPC A61F 2/246; A61F 2/2454; A61F 2/2466; A61F 2220/0075

(57) ABSTRACT

A clip or clamping devices for use in shaping a valve (such as, for example, the aortic valve) to address valvular malfunction. Each clip is implanted into the valve by positioning the clip over a commissure to partially or completely close an interleaflet triangle. Each clip has arms, a blade or paddle disposed at the distal end of each arm, rings attached along an outer surface of each of the arms, and a proximal attachment mechanism attached at a proximal end of the clip. Each clip can have a tension component configured to urge the two arms together into a clamped configuration. Each clip can be introduced by catheter and can be manipulated via strings and guide wires.

### CROSS-REFERENCE TO RELATED APPLICATION(S)

This application claims priority to U.S. Provisional Application No. 63/164,701 filed Mar. 23, 2021 and entitled “Valve Reshaping Device, System, and Related Methods,” which is hereby incorporated by reference in its entirety under 35 U.S.C. § 119(e).

### TECHNICAL FIELD

The disclosure relates to various medical devices, systems and methods of use. Namely, an aortic valve device, namely, a clip that is implanted into the valve by positioning the clip over a commissure to partially or completely close an interleaflet triangle.

### BACKGROUND

As shown in FIG. 1A, the human heart has four chambers and four valves, including the aortic valve 10. A valve opens and shuts to allow blood to pass from each chamber of the heart and prevent the backward flow of that blood.

As best shown in FIGS. 1B and 1C, the components of the aortic valve 10 include: the sinotubular junction 12, the ventriculo-aortic junction (or “basal annulus”) 14, the interleaflet triangles 16, and, as best shown in FIG. 1A, the leaflets (or “cusps”) 18. Each cusp 18 has a margin where it is attached to the aortic annulus in a semilunar fashion. The semilunar hinge-lines of adjacent leaflets 18 meet at the level of the sinotubular junction 12, thereby forming what is known as the commissures 20, such that the plane connecting the three commissures 20 forms the sinotubular junction 12. As best shown in FIG. 1A, each cusp 18 has 2 free edges, each adjacent to and in contact with two adjacent cusps 18. Further, at the center of the free edges, there is the nodule of Arantius 22, which is a small fibrous bulge. The free edges (or rims) of each of the cusps 18 are thicker than the cusp body. As a result, during valve closure, the overlap of these rims of adjacent cusps 18 serve to increase the valve support. When the valve is closed, each of the 3 valve cusps contain a sinus 24 called the “sinus of Valsalva” or “aortic sinus.” The sinuses of Valsalva 24 are outpouching aortic wall structures that are demarcated by the insertion of each cusp. The width of the sinuses 24 is more than the left ventricular outflow tract and of the ascending and when the valve opens, the leaflets fall back into their sinuses 24.

The body of each leaflet 18 is thin and pliable but has a core of fibrous tissue with endothelial linings on its arterial and ventricular sides. During systole, or the phase of ventricular contraction of the cardiac cycle, the pressure in the left ventricle is increased and the aortic valve leaflets 18 are pushed apart and they fall back into their respective sinuses 24, allowing ejection of blood into the aortic root with no impediment on coronary flow. During diastole, or the relaxation phase of the ventricle, the pressure in the ventricle drops below that in the aortic root and the aortic valve leaflets 18 close, thereby preventing regurgitation of the blood into the left ventricle. In a normal aortic valve, the adjacent cusps 18 coapt (coaptation height) by at least 2 mm to ensure valve competence (normal length of coaptations ranges from 2-6 mm). There are multiple other parameters that describe cusp anatomy and function including geometric height, effective height, and commissure height.

The normal anatomy of the aortic valve 10 is tricuspid, meaning it has 3 cusps or leaflets 18, but a common cardiac valvular anomaly is a bicuspid aortic valve (with 2 leaflets), occurring in 1-2% of the general population. A bicuspid aortic valve results from fusion of aortic valve leaflets and occurs most commonly (≈80%) between the right coronary and left coronary leaflets with secondary association with future complications such as insufficiency and/or stenosis.

The interleaflet triangles (also known as subcommisural triangles) 16 mentioned above are important in the normal physiology and hemodynamics of the aortic valve and represent important interrelationships between the aortic sinuses 24, the leaflets 18, and supporting left ventricular structures. The 3 interleaflet triangles 16 are delineated superiorly by the commissures 20, laterally by the attachment of the two adjacent leaflets 18 to the annulus and the left ventricular wall, and inferiorly by basal annulus 14.

There are two typical forms of valve malfunction (which can exhibit themselves separately or in combination in patients): (1) insufficiency (leakage of the valve); and (2) stenosis (narrowing of the valve).

Aortic valve insufficiency (AI) or regurgitation is a condition in which the valve doesn't close properly, leading to blood flowing backwards in the heart (back into the left ventricle) during diastole instead of pumping out to the body organs. The heart compensates by pumping harder, which, over time, can lead to weakening of the heart muscle and ultimately heart failure.

The prevalence of AI increases with advancing age, and the prevalence of moderate or severe AI has been estimated to be 1.6% of individuals ≥65 years. AI can result from different pathophysiologic mechanisms, including a dilated aortic annulus, single cusp prolapse in tricuspid aortic valves, or conjoined cusp prolapse in bicuspid aortic valves. The dilated aortic annulus results in a sagging of the belly of the cusps resulting in lack of central cusp apposition.

In patients with chronic AI, the degree of sino-tubular junction and ventriculo-aortic junction dilatation correlates with the severity of AI by preventing adequate cusp coaptation.

Conventional treatment for such a condition is surgery with replacement of the aortic valve using either a prosthetic mechanical valve or prosthetic tissue valve. Disadvantages of aortic valve replacement include (1) possible late prosthetic valve dysfunction secondary to structural failure, (2) bacterial infection of the prosthesis, and (3) the need to keep the patient on blood thinners.

One solution has been the development of aortic valve repair methods to restore normal function of the aortic valve (instead of replacement). Such known methods have exhibited safe and excellent immediate results with encouraging durability with up to 15-20 years of follow-up.

For example, one known aortic valve repair procedure is the reduction annuloplasty, which, as shown in FIGS. 2A and 2B, involves closure of the three interleaflet or subcommissural triangles by horizontal mattress sutures 30 reinforced with teflon pledgets 32. This closure leads to increasing the surface area of cusp coaptation and subsequently central regurgitation. The stitch is placed at 50% of the interleaflet triangle height, allowing optimal leaflet coaptation and regurgitation orifice area reduction without impinging blood flow through the ventriculo-aortic junction. In the upper half of the interleaflet triangle, the leaflets on the two sides are almost parallel, while in their lower half, the leaflets start to diverge to reach the nadir of the correspondent cusp. Closing the upper part with a pledget-reinforced braided suture increases the co-aptation without significantly impinging leaflet motion and doesn't induce significant alterations in the hydrodynamics of the aortic root.

In a bicuspid aortic valve, regurgitation results from the prolapse of the conjoint cusp. Surgical repair in these cases include resection of the redundant free margin in the central fused raphe portion in addition to closure of the two subcommissural triangles as described above. In cusp prolapse in a tricuspid valve, AI is caused by the prolapse of one or more cusps. This is repaired surgically by resection to shorten the free margin to meet the other cusps triangular resection in the center of the cusp with re-approximation of the cut edges of the cusp with interrupted 6-0 polypropylene sutures and closure of the three subcommissural triangles as previously described.

Recently, a less invasive procedure has been introduced to treat the narrowing of the aortic valve (aortic valve stenosis): transcatheter aortic valve implantation (TAVI). This method has proved to be as effective, and safer, than the traditional surgical replacement. There have been more than 200,000 patients treated with TAVI in the US alone.

However, in the case of pure native AI, TAVI is not considered a safe alternative for surgical replacement or repair because of the absence of significant leaflet or annular calcifications in most cases of pure AI. The positioning of the prosthesis in TAVI in the precisely correct position depends on anchoring on the calcifications universally found in cases of aortic valve stenosis. Thus, TAVI for patients with pure AI carries potential risks including (1) malpositioning due to inadequate sealing, (2) valve embolization, and (3) significant leak around the valve (paravalular regurgitation). Oversizing of the TAVI prosthesis in an attempt to better anchor and seal the device also involves a risk of valve dislocation, conduction disorders, and annulus rupture.

To date, there have been no known or reported non-invasive methods or systems to repair the aortic valve in cases of AI. Even though standard surgical repair of the valve is an established effective method, it is a very invasive method with several risks including death, stroke, bleeding, infection, heart rhythm problems, blood clots, significant discomfort, an extended hospital stay, and prolonged recovery. Also, it has been estimated that 7.8% of patients with severe AI, who needed a surgical intervention, did not get treated mainly because of advanced age and multiple comorbidities making surgery of excessive risk.

One known system for repairing a valve is disclosed in U.S. Published Application 2004/0199183, which provides for a grasper tool that is used to deploy a fastener for attachment to the leaflets of the valve. One disadvantage of the system is the need for the grasper, which increases the size requirements for the entire system and the complexity thereof.

There is a need in the art for an improved device and related non-invasive or less-invasive methods for aortic valve repair in the treatment of AI.

### BRIEF SUMMARY

Described herein are various implementations relating to devices, systems and methods for an improved device and related non-invasive or less-invasive methods for aortic valve repair in the treatment of AI.

In Example 1, a valve clamping device, the device comprising a first arm comprising at least one first string attachment structure operably coupled to the first arm and a first tissue contact structure operably coupled to the first arm at or near the distal end of the first arm a second arm operably coupled at a proximal end to a proximal end of the first arm, the second arm comprising at least one second string attachment structure operably coupled to the second arm and a second tissue contact structure operably coupled to the second arm at or near the distal end of the second arm a proximal attachment mechanism operably coupled to the proximal ends of the first and second arms and an arm coupling device attached to the first and second arms.

In Example 2, the valve clamping device of Example 1, wherein the arm coupling device comprises a tension mechanism or a locking mechanism.

In Example 3, the valve clamping device of Example 2, wherein the arm coupling device comprises the tension mechanism, wherein the tension mechanism is a tension spring.

In Example 4, the valve clamping device of Example 2, wherein the arm coupling device comprises the locking mechanism, wherein the locking mechanism comprises a locking rod coupled at a first end to the first arm and at a second end to the second arm and an attachment structure associated with the locking rod.

In Example 5, the valve clamping device of Example 4, further comprising an elongate actuation structure coupleable to the attachment structure.

In Example 6, the valve clamping device of Example 1, further comprising a first actuation string disposed through the at least one first string attachment structure and a second actuation string disposed through the at least one second string attachment structure.

In Example 7, the valve clamping device of Example 1, wherein the first and second arms are movable in relation to each other between an open configuration and a closed configuration in which the first and second tissue contact structures are disposed in close proximity.

In Example 8, the valve clamping device of Example 1, wherein the first and second tissue contact structures comprise attachment enhancement mechanisms disposed thereon.

In Example 9, the valve clamping device of Example 8, wherein the attachment enhancement mechanisms comprises spikes.

In Example 10, the valve clamping device of Example 1, wherein the proximal attachment mechanism is coupleable to a delivery catheter.

In Example 11, the valve clamping device of Example 1, wherein the device is sized to be positionable through a delivery sheath.

In Example 12, a valve clamping device, comprising a plurality of arms having proximal and distal ends a plurality of blades or paddles disposed at the distal ends of the plurality of arms a plurality of rings attached to the plurality of arms and a proximal attachment mechanism.

In Example 13, the valve clamping device of Example 12, wherein the proximal attachment mechanism is a knob configured to be attached to a delivery device used in conjunction with the valve clamping device.

In Example 14, the valve clamping device of Example 13, wherein the knob is threaded.

In Example 15, the valve clamping device of Example 12, further comprising a tension component configured for tensioned and untensioned states.

In Example 16, the valve clamping device of Example 12, further comprising a central rod defining a rod lumen.

In Example 17, the valve clamping device of Example 16, wherein the rod lumen is sized to accommodate a guide wire.

In Example 18, a valve clamping device, the device comprising a first arm comprising at least one first string attachment structure operably coupled to the first arm and a first tissue contact structure operably coupled to the first arm at or near the distal end of the first arm a second arm operably coupled at a proximal end to a proximal end of the first arm, the second arm comprising at least one second string attachment structure operably coupled to the second arm and a second tissue contact structure operably coupled to the second arm at or near the distal end of the second arm.

In Example 19, the valve clamping device of Example 18, further comprising a proximal attachment mechanism operably coupled to the proximal ends of the first and second arms.

In Example 20, the valve clamping device of Example 18, further comprising an arm coupling device attached to the first and second arms. While multiple implementations are disclosed, still other implementations of the disclosure will become apparent to those skilled in the art from the following detailed description, which shows and describes illustrative implementations of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1A is a perspective view of a human heart showing the aortic valve and cusps.

FIG. 1B is a perspective, transparent schematic of the aortic valve showing the internal structure.

FIG. 10 is a further perspective, transparent schematic of the aortic valve showing the interleaflet triangles.

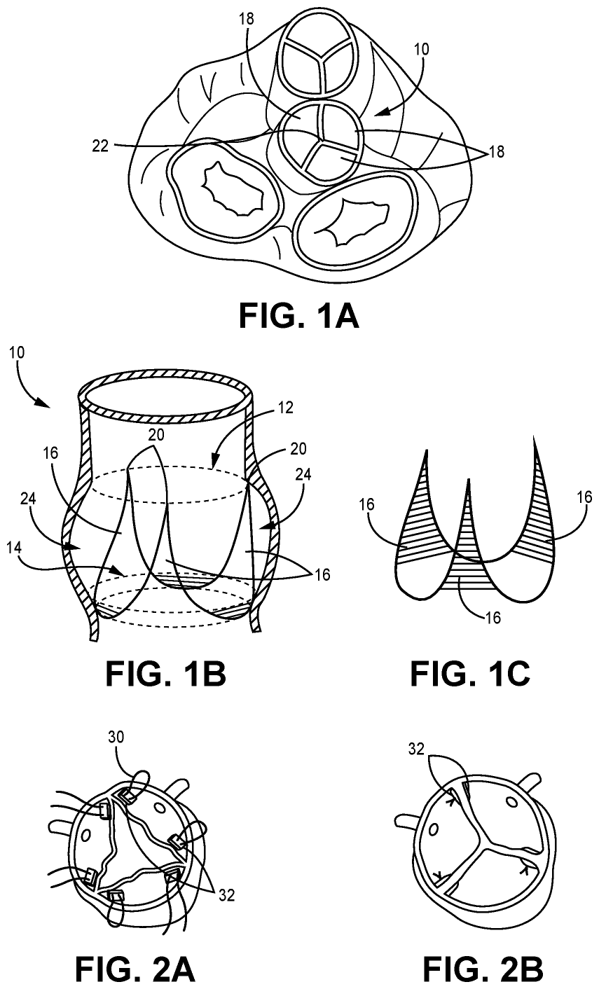

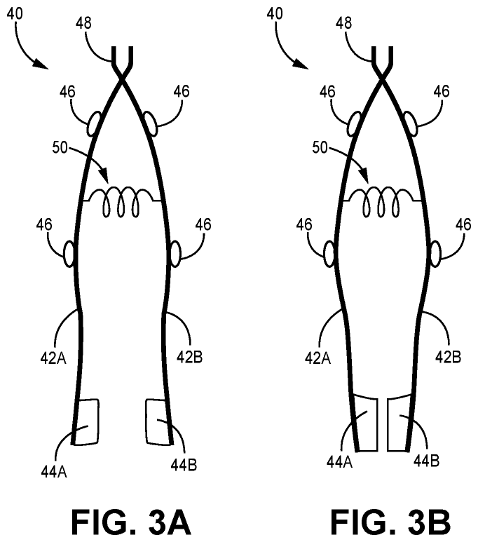

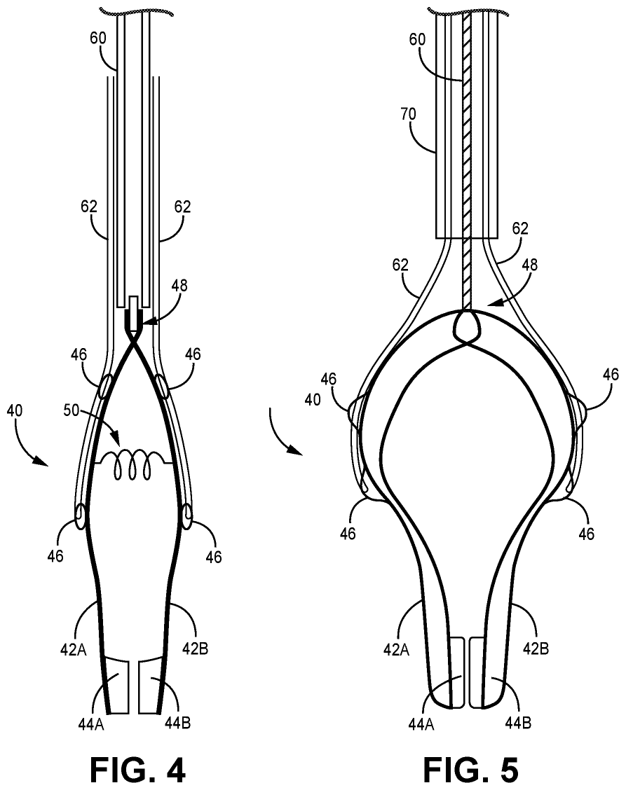

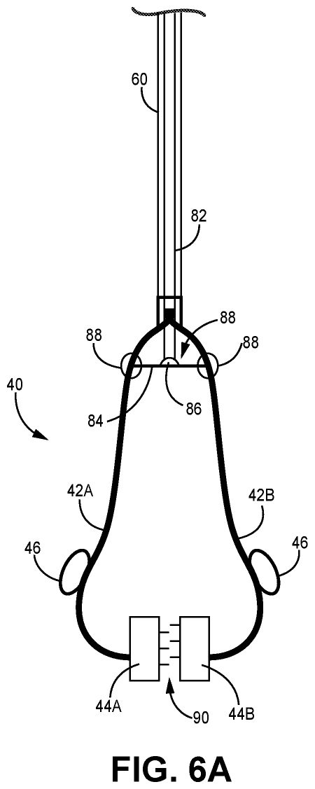

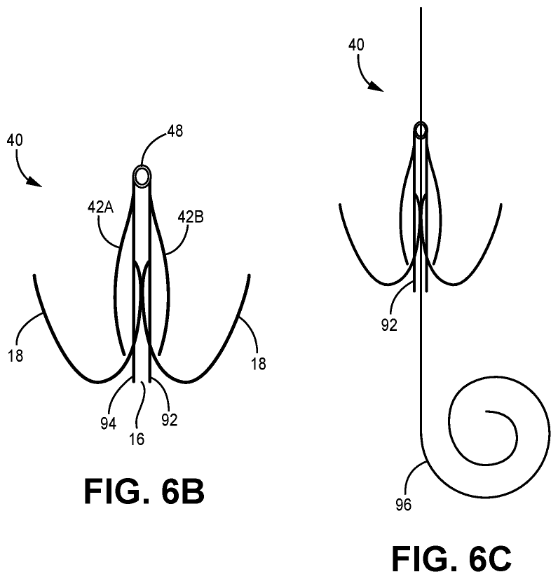

FIG. 2A is a head-on view of an aortic valve undergoing eduction annuloplasty.

FIG. 2B is a further head-on view of an aortic valve undergoing eduction annuloplasty.

FIG. 3A is a schematic side view of the clip device in an open or unclamped configuration, according to one implementation.

FIG. 3B is a schematic side view of the clip device in a closed or clamped configuration, according to one implementation.

FIG. 4 is a schematic side view of the clip device in use with a delivery catheter, according to one implementation.

FIG. 5 is a schematic side view of the clip device in use with a sheath, according to one implementation.

FIG. 6A is a schematic side view of the clip device according to one implementation having spikes or pins disposed on each of the paddles.

FIG. 6B is a schematic side view of the clip device according to one implementation having a central rod comprising a lumen 94 therethrough that can allow for the introduction of a guide wire.

FIG. 6C is a further view of the implementation of FIG. 6B with a guide wire disposed through the lumen.

FIG. 7 is a schematic side view of the clip device according to one implementation having a single clip that is positioned over and clamped to a first leaflet and an adjacent second leaflet.

FIG. 8A is a schematic depiction of the use of three clips in the open or unclamped position, with each clip disposed over a different one of the three commissures of an aortic valve.

FIG. 8B is a further schematic depiction of the use of three clips as in FIG. 8A with the clips in the clamped or closed position.

FIG. 9A depicts a further schematic view of three clips positioned over the three target commissures of an aortic valve.

FIG. 9B depicts a further schematic view of three clips of FIG. 9A in the closed or clamped position.

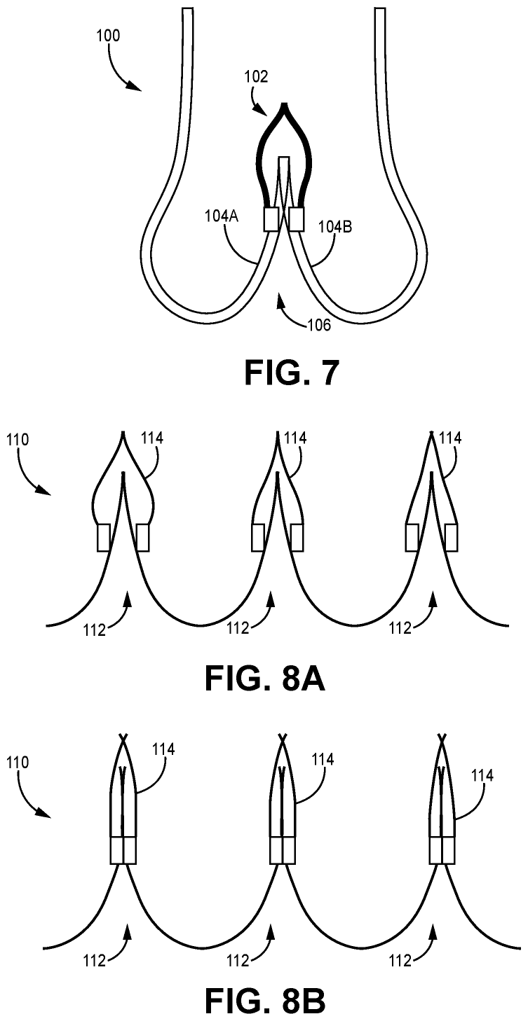

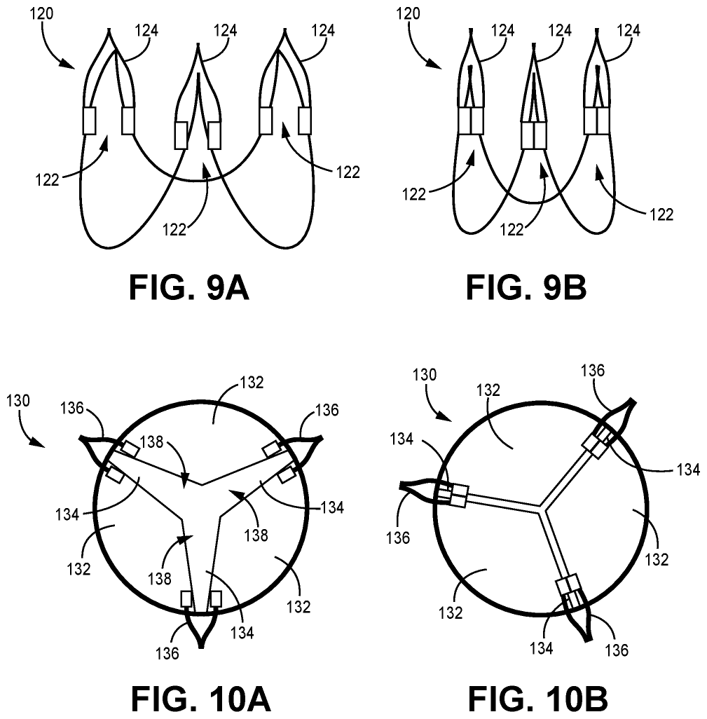

FIG. 10A depicts a top schematic view of three clips in their open configurations and positioned over the target commissures such that there are gaps between the leaflets.

FIG. 10B depicts the implementation of FIG. 10A wherein the devices or clips are in the closed or clamped configuration.

### DETAILED DESCRIPTION

The various implementations herein relate to one or more clip or clamping devices for use in shaping a valve (such as, for example, the aortic valve) to address valvular malfunction. According to certain implementations, these clips or clamping devices can be implanted in a minimally invasive manner using a catheter, guide wire or the like. These various implementations serve as a repair mechanism for a leaky valve (such as an aortic valve) as a result of valve insufficiency or regurgitation and/or as anchors for future transcutaneous valve implantation. In use according to certain implementations, each clip is implanted into the valve by positioning the clip over a commissure to partially or completely close an interleaflet triangle. In most cases, three clips are used, with each clip being positioned over one of the three commissures.

Unlike the system discussed above which is disclosed in U.S. Published Application 2004/0199183, no separate grasper tool is required for deployment of the fastening device. Instead, the various implementations described herein relate to deployable fastening or clip devices that can be implanted without the need for a grasper device or any kind of separate device with clamping components.

The various implementations herein provide less invasive methods and systems for repair of valves and treatment of valvular insufficiency or regurgitation. In certain implementations, the clip implementations herein induce reshaping of the three interleaflet triangles (or subcommissural triangles) of a semilunar valve (such as the aortic valve, for example). The procedures performed using the various clip implementations herein can also be described as a percutaneous subcommissural annuloplasty, which consists of constriction of the interleaflet triangles of the aortic valve. Further, the various clip implementations can also be used to adjust the interleaflet triangle by closing it at a prespecified height. This adjustment can restore the coaptation of the three leaflets of the valve, thereby decreasing regurgitation.

One implementation of a clip 40 or clamping device 40 is depicted in FIGS. 3A and 3B. The clip 40 has two arms 42A, 42B, each having a tissue contact structure 44A, 44B such as a blade or paddle 44A, 44B disposed at the distal end of each arm 42A, 42B, as well as string attachment mechanisms 46 such as rings 46 attached along an outer surface of each of the arms 42A, 42B, and a proximal attachment mechanism 48 attached at a proximal end of the clip 40.

In addition, the clip 40 of these implementations and others has a tension component 50 disposed between and attached to each of the arms 42A, 42B such that when the tension component 50 is in its untensioned state, the two arms 42A, 42B are disposed in their clamped or closed configuration as best shown in FIG. 3B. According to one implementation, the tension component 50 is a spring 50. Alternatively, the tension component 50 can be any known selectively tensioned mechanism. Regardless of the specific mechanism, the tension component 50 is configured to urge the two arms 42A, 42B together into a clamped configuration.

In accordance with some implementations, the proximal attachment mechanism 48 can be a knob 48 that is attached to any delivery device used in conjunction with the clip 40. The knob 48 can be attached in a threaded manner or alternatively any known manner. If a threaded mechanism is used, the clip 40 can be secured to the catheter or other delivery device by rotating such proximal attachment mechanism 48 clockwise, and the clip 40 can be removed by rotating the device in the counterclockwise direction. In a further alternative, the attachment mechanism 48 can be any known mechanism for attachment to a delivery device.

The arms 42A, 42B can be plates or wires of varying width or diameter. Alternatively, the arms 42A, 42B can have any known shape or configuration. In addition, the arms 42A, 42B can vary in length depending on the anatomical and functional characteristics of the target valve. In various implementations, the arms 42A, 42B, and alternatively the entire clip 40, can be covered in a polymeric material such as PTFE. Alternatively, the material can be any known material that promotes the endothelization and covering of the device 40 with body tissue.

The arms 42A, 42B can be made of any known shape memory material. For example, in certain implementations the arms 42A, 42B can be made of nitinol. Alternatively, the arms 42A, 42B can be made of another metal such as Elgiloy, Phynox, titanium, a titanium alloy, or a stainless steel alloy. In a further alternative, any known shape memory metal or other material can be used. Regardless of the material, according to certain implementations, the material should provide shape memory and sufficient recoil force to hold two adjacent leaflets together in an aortic valve.

The blades or paddles 44A, 44B are configured to be the contact points for the valvular tissue. More specifically, the paddles 44A, 44B are disposed at the distal end of the arms 42A, 42B such that when the arms 42A, 42B are in the clamped configuration, the paddles 44A, 44B are urged together, thereby urging the tissue of the two adjacent leaflets together such that the leaflet tissue is clamped between the two paddles 44A, 44B.

The paddles 44A, 44B are configured to enhance contact with and attachment to the leaflet tissue. That is, the paddles 44A, 44B can be circular, rectangular, square, or any other known shape of any appropriate size. The paddles 44A, 44B, in certain implementations, can be covered with a polymeric material such as PTFE. Further, in certain implementations, the paddles 44A, 44B can have spikes, pins, or needles that are urged into the target leaflet tissue and thereby improve stability of the clip 40 and attachment to the tissue. Such spikes will be discussed in additional detail below.

Any components of any of the subsequent clip implementations can have the same or similar functions and/or features as the components described above with respect to the implementation depicted in FIGS. 3A and 3B. Further, any of the features and/or functions of any subsequent implementations can be incorporated into any other implementation, including the above implementation.

According to certain implementations, the clip 40 is movable between an open configuration and a closed configuration. In this particular exemplary implementation, FIG. 3A depicts the clip 40 in its open configuration, while FIG. 3B shows the clip 40 in its closed or clamped configuration in which the paddles 44A, 44B are in contact or close proximity. As mentioned above, the tension component 50 is configured to urge the two arms 42A, 42B together into the closed or clamped configuration. More specifically, the tension component 50 is shown in FIG. 3B in its relaxed state. Thus, the arms 42A, 42B must be urged apart by some external force to move the arms 42A, 42B into the open configuration of FIG. 3A. As the arms 42A, 42B are urged apart, the tension component 50 is urged into a tensioned state as shown in FIG. 3A. This means that when the external force is removed from the arms 42A, 42B, the tension component 50 will urge the arms 42A, 42B back into the closed or clamped configuration of FIG. 3B.

As mentioned above, according to certain implementations, each clip can be delivered to the patient's aortic valve via a minimally invasive procedure using a catheter and/or sheath. According to one specific implementation as shown in FIG. 4, the clip 40 is attached at the proximal attachment structure 48 to a delivery catheter 60. Further, in this implementation, strings (or other similar elongate components) 62 are threaded through the rings 46 on each side of the clip 40 such that a first string 62 is threaded through the rings 46 of the first arm 42A, and a second string 62 is threaded through the rings 46 of the second arm 42B, with both strings extending proximally out of the patient such that the proximal ends of the strings are accessible by a surgeon or other user. As such, the clip 40 can be delivered to the target aortic valve via the catheter 60 such that the catheter is advanced in a minimally invasive manner through a blood vessel of the patient until the clip 40 is positioned as desired.

In use, when the clip 40 is positioned near the target commissure, the threads 62 can be urged in a proximal direction by a surgeon or other user, thereby urging the arms 42A, 42B apart, resulting in the clip 40 been urged into its open configuration (similar to FIG. 3A, for example). In this configuration, the clip 40 is advanced by the catheter 60 into position over the target commissure, and the force is removed from the threads 62, thereby allowing the tension mechanism 50 to urge the arms 42A, 42B back into the closed position over the adjacent leaflets.

In accordance with a further implementation and as shown in FIG. 5, a sheath 70 can also be used in conjunction with the catheter 60 to deliver the clip 40. In this implementation, the catheter 60 is disposed through the sheath and still attached to the clip 40 at the proximal attachment mechanism 48. Further, the proximal ends of both sets of strings 62 that are threaded through the rings 46 are also disposed through the sheath 70. In use, the clip 40 is delivered to the target aortic valve via the catheter 60 disposed through the sheath 70 such that both the catheter 60 and the sheath 70 are advanced through the blood vessel with the clip 40 disposed on the distal end of the catheter 60. The operation of the clip for purposes of positioning it over a target commissure and clamping it thereto is substantially similar to the steps described above. The sheath 70 would further help in properly orienting and delivering the catheter 60 and the clip 40 to the target position.

In yet another implementation as depicted in FIG. 6A, the clip 40 can have the same or similar components to the various clip implementations discussed above, along with a locking mechanism 80. The locking mechanism 80 can be disposed between the two arms 42A, 42B and further can be attached to an elongate actuation structure 82 moveably disposed within the catheter 60. In accordance with one implementation, the locking mechanism 80 has a rod 84, with an attachment mechanism 86 disposed thereon such that the elongate actuation structure 82 is attached to the rod 84 at the attachment mechanism 86.

Further, the rod 84 can have rod attachment rings or mechanisms 88 attached to each of the arms 42A, 42B such that the rod 84 is coupled to the arms 42A, 42B via the rod attachment rings 88. As such, actuation of the locking mechanism 80 by the elongate actuation structure 82 can cause force to be applied to the locking mechanism 80 at the attachment mechanism 86, thereby actuating the mechanism 82 to lock the two arms 42A, 42B in place.

For example, in one specific implementation, the actuation structure 82 can be urged distally to actuate the locking mechanism 80 to lock the two arms 42A, 42B. Further, once the two arms 42A, 42B are locked in place, the actuation structure 82 can be rotated counter-clockwise to detach from the attachment mechanism 86 and then can be retracted proximally out of the body. According to certain implementations, the elongate actuation structure 82 can be a catheter, wire, or any other known structure for use in actuating a mechanism such as the locking mechanism 80.

In use, according to one implementation, advancing the actuation structure 82 causes the locking mechanism 80 to lock or further tighten the two arms 42A, 42B together, while retracting the actuation structure 82 will loosen the locking mechanism 80 and thereby release the arms 42A, 42B. Alternatively, the locking mechanism 80 as shown can be operated in any known fashion. In a further alternative, any known locking or tightening mechanism can be used.

In addition, the clip 40 in this particular implementation as shown in FIG. 6A has spikes or pins 90 disposed on each of the paddles 44A, 44B. These spikes 90 can enhance the attachment of the paddles 44A, 44B to the leaflet tissue without causing damage or tearing of that tissue. Alternatively, any additional attachment enhancement mechanisms or features can be incorporated into the paddles 44A, 44B.

Any of the other implementations disclosed or contemplated herein can have a locking mechanism similar to the mechanism 80 described above and/or spikes or pins in the paddles similar to the spikes or pins 90 described above.

In yet another implementation as depicted in FIG. 6B, the clip 40 can have the same or similar components to the various clip implementations discussed above, along with a central rod 92 comprising a lumen 94 therethrough that can allow for the introduction of a guide wire 96 (shown in FIG. 6C) for use in introduction and manipulation of the clip 40, as would be readily appreciated.

In use, any of the known clip implementations and related catheter and/or sheath implementations can be delivered to the target valve via cardiovascular access. More specifically, the clip and delivery device can be delivered via the femoral artery, radial artery, brachial artery, axillary artery, carotid artery, or any other similar artery. Further, in certain implementations, the delivery can be accomplished using visual guidance such as fluoroscopy and/or ultrasound technologies. Further, in certain implementations the delivery can be accomplished via other physical introduction technologies such as via a guide wire.

FIGS. 7 through 10B depict the use of one or more clips to modify the shape of a valve. More specifically, in these specific examples, the valve is an aortic valve. For example, as shown in FIG. 7, a single clip 102 is positioned over and clamped to a first leaflet 104A and an adjacent second leaflet 104B. As shown, the use of the clip 102 reduces the size of the interleaflet triangle 106. More specifically, the space between the two leaflets 104A, 104B is reduced by the use of the clip 102. AIl of the various clip implementations as described above and below can be used to clamp together two adjacent leaflets, thereby reducing the size of the interleaflet triangle, and thus causing reduction of the circumferential diameter of the aortic annulus. Further, the clip can improve the coaptation of the adjacent leaflets by increasing the contact and/or overlap of the free edges of those leaflets. The extent of the reduction in the width of the interleaflet triangle that is achieved by the clip will depend on the closure force exerted by the clip, the positioning of the clip (the depth of the implant and/or the distance from the aortic wall), and the shape and dimensions of the clip arms and paddles.

FIGS. 8A and 8B provide a schematic depiction of the use of three clips 114, with each clip disposed over a different one of the three commissures of an aortic valve 110. More specifically, FIG. 8 depicts three clips 114 in their open configurations and positioned over the three commissures as shown. Further, FIG. 8B depicts those same three clips 114 in their clamped configurations, thereby urging together the adjacent leaflets such that the interleaflet triangles 112 are reduced in size.

In contrast to FIGS. 8A and 8B, FIGS. 9A and 9B depict a more accurate view of three clips 124 positioned over the three target commissures of an aortic valve 120, because the aortic valve 120 in these figures is in its circular shape. FIG. 9A depicts the three clips 124 in their open configuration adjacent to the target valve 120 prior to clamping the adjacent leaflets together. As such, the interleaflet triangles 122 in FIG. 9A remain unrestricted. In contrast, FIG. 9B depicts the clips 124 in their closed configurations after they have been positioned as desired over the commissures, thereby urging the adjacent leaflets together and reducing the size of the interleaflet triangles 122.

Finally, FIGS. 10A and 10B depict a top view of a similar process. More specifically, FIG. 10A depicts three clips 136 in their open configurations and positioned over the target commissures 134 such that there are gaps 138 between the leaflets 132. These gaps 138 can be the result of any of the valve malfunctions described above. Thus, as shown in FIG. 10B, when the clips 136 are positioned over the target commissures and actuated to move into their closed or clamped configurations as shown, this results in the adjacent leaflets 132 being urged together such that the commissural lines are close together such that the gaps are reduced or eliminated and thus the valve malfunction is corrected.

In any of the various implementations described above, the various clip implementations can be used in a variety of ways to treat a malfunctioning valve. For example, a clip can be implanted to prevent the prolapse of one of two adjacent leaflets by attaching the free edge of the prolapse and leaflet to the free edge of the normal leaflet and thus prevent the redundant leaflet from dropping below the margin of normal valve closure. Additionally, a clip can be used to approximate the adjacent leaflets in cases where gaps have formed secondary to dilation of the annulus of the valve. Further, as discussed above, a clip can be positioned adjacent to the commissural line or alternatively at a more central location depending on the anatomical and functional characteristics of the valve. The depth of the clip into the sinuses can vary depending on the anatomical and functional characteristics of the valve. According to further implementations, one or more clips could be placed at varying distances between each other along each specific line of coaptation between two adjacent leaflets. Further, such clips can be placed on any or all of the coaptation lines between the two adjacent leaflets.

According to various implementations, any clip implementation herein can be recaptured after initial implantation and repositioned depending on the anatomical and functional needs before it is fully released by the delivery device. In further implementations, any clip or clips positioned adjacent to the commissural lines can be used as anchors or docking mechanisms to help stabilize the placement of a transcatheter aortic valve implantation device.

While multiple implementations are disclosed, still other implementations will become apparent to those skilled in the art from the following detailed description, which shows and describes illustrative implementations. As will be realized, the various implementations are capable of modifications in various obvious aspects, all without departing from the spirit and scope thereof. Accordingly, the drawings and detailed description are to be regarded as illustrative in nature and not restrictive.

Although the various implementations have been described with reference to preferred implementations, persons skilled in the art will recognize that changes may be made in form and detail without departing from the spirit and scope thereof.

What is claimed is:

1. A valve clamping device, the device comprising:
(a) a first arm comprising:
(i) at least one first string attachment structure operably coupled to the first arm; and
(ii) a first tissue contact structure operably coupled to the first arm at or near the distal end of the first arm;
(b) a second arm operably coupled at a proximal end to a proximal end of the first arm, the second arm comprising:
(i) at least one second string attachment structure operably coupled to the second arm; and
(ii) a second tissue contact structure operably coupled to the second arm at or near the distal end of the second arm;
(c) a proximal attachment mechanism operably coupled to the proximal ends of the first and second arms; and
(d) an arm coupling device attached to the first and second arms.

2. The valve clamping device of claim 1, wherein the arm coupling device comprises a tension mechanism or a locking mechanism.

3. The valve clamping device of claim 2, wherein the arm coupling device comprises the tension mechanism, wherein the tension mechanism is a tension spring.

4. The valve clamping device of claim 2, wherein the arm coupling device comprises the locking mechanism, wherein the locking mechanism comprises:
(a) a locking rod coupled at a first end to the first arm and at a second end to the second arm; and
(b) an attachment structure associated with the locking rod.

5. The valve clamping device of claim 4, further comprising an elongate actuation structure coupleable to the attachment structure.

6. The valve clamping device of claim 1, further comprising a first actuation string disposed through the at least one first string attachment structure and a second actuation string disposed through the at least one second string attachment structure.

7. The valve clamping device of claim 1, wherein the first and second arms are movable in relation to each other between an open configuration and a closed configuration in which the first and second tissue contact structures are disposed in close proximity.

8. The valve clamping device of claim 1, wherein the first and second tissue contact structures comprise attachment enhancement mechanisms disposed thereon.

9. The valve clamping device of claim 8, wherein the attachment enhancement mechanisms comprises spikes.

10. The valve clamping device of claim 1, wherein the proximal attachment mechanism is coupleable to a delivery catheter.

11. The valve clamping device of claim 1, wherein the device is sized to be positionable through a delivery sheath.

12. A valve clamping device, comprising:
(a) a plurality of arms having proximal and distal ends;
(b) a plurality of blades or paddles disposed at the distal ends of the plurality of arms;
(c) a plurality of rings attached to the plurality of arms; and
(d) a proximal attachment mechanism.

13. The valve clamping device of claim 12, wherein the proximal attachment mechanism is a knob configured to be attached to a delivery device used in conjunction with the valve clamping device.

14. The valve clamping device of claim 13, wherein the knob is threaded.

15. The valve clamping device of claim 12, further comprising a tension component configured for tensioned and untensioned states.

16. The valve clamping device of claim 12, further comprising a central rod defining a rod lumen.

17. The valve clamping device of claim 16, wherein the rod lumen is sized to accommodate a guide wire.

18. A valve clamping device, the device comprising:
(a) a first arm comprising:
(i) at least one first string attachment structure operably coupled to the first arm; and
(ii) a first tissue contact structure operably coupled to the first arm at or near the distal end of the first arm;
(b) a second arm operably coupled at a proximal end to a proximal end of the first arm, the second arm comprising:
(i) at least one second string attachment structure operably coupled to the second arm; and
(ii) a second tissue contact structure operably coupled to the second arm at or near the distal end of the second arm.

19. The valve clamping device of claim 18, further comprising a proximal attachment mechanism operably coupled to the proximal ends of the first and second arms.

20. The valve clamping device of claim 18, further comprising an arm coupling device attached to the first and second arms.
